(19) United States
(12) Patent Application Publication
(10) Pub. No.: US 2026/0188422 A1
(43) Pub. Date: Jul. 2, 2026

(54) METHOD OF GENERATING NEW DRUG CANDIDATE INFORMATION USING VIRTUAL MOLECULAR MODELING OF PROTEIN POCKET STRUCTURE

(71) Applicant: NAMUICT CO., LTD., Suwon-si (KR)

(72) Inventors: Min Sun YEOM, Seoul (KR); Hye Ree YOON, Seoul (KR)

(21) Appl. No.: 19/093,229

(22) Filed: Mar. 27, 2025

(30) Foreign Application Priority Data
Dec. 30, 2024 (KR) 10-2024-0200727

(51) Int. Cl. G16B 15/30; G16B 15/20; G16B 40/20

(52) U.S. Cl. CPC G16B 15/30; G16B 15/20; G16B 40/20

(57) ABSTRACT

Disclosed is a method of generating new drug candidate information using virtual molecular modeling of a protein pocket structure, including identifying hit compounds having selective ligand characteristics only for a target protein, which is a cause of pathogenesis present in cells, from existing compounds using virtual molecular modeling of a protein pocket structure and artificial intelligence, generating information on novel compounds having ligand characteristics similar to the hit compounds for the target protein, and determining, among the novel compounds, novel compounds having predicted binding affinity for the target protein and in vitro properties and in vivo properties that meet preset criteria as new drug candidates, thus generating new drug candidate information, thereby enabling effective development of new drugs with relatively low R&D costs and short development time, the method including a hit compound search step (S100), an analogue/lead compound search step (S200), and a new drug search step (S300).

### BACKGROUND OF THE INVENTION

### Field of the Invention

The present invention relates to a method of generating new drug candidate information using virtual molecular modeling of a protein pocket structure, and more particularly, to a method of generating new drug candidate information, which includes identifying hit compounds having selective ligand characteristics only for a target protein, which is a cause of pathogenesis present in cells, from existing compounds using virtual molecular modeling of a protein pocket structure and artificial intelligence, generating information on novel compounds having ligand characteristics similar to the hit compounds for the target protein, and determining, among the novel compounds, novel compounds having predicted binding affinity for the target protein and in vitro properties and in vivo properties that meet preset criteria as new drug candidates.

### Description of the Related Art

In modern society, the lives of citizens are becoming healthier and more comfortable and the average life expectancy is increasing significantly due to the development of scientific technology and social systems that enable a clean and sanitary life, for example, the construction of social overhead capital such as electricity, communications, water and sewage systems, roads, ports, and airports, the expansion of social systems such as national health insurance, national pension, and compulsory education, and the development of medical technology.

However, despite the fact that incurable diseases are gradually being conquered by virtue of advances in medicine, there are still some diseases that are difficult to treat even with modern medicine and are fatal, leading to death when contracted.

In particular, various new drugs are being developed for the treatment of diseases, but new drug development is a high-risk, high-return industry, and a huge amount of time and money is required to develop new drugs. It takes an average of 10 to 12 years from the beginning of the new drug development process to launching a new drug product, and it is estimated that the average development cost per new drug is 2.168 billion dollars. The huge time and cost required for new drug development acts as an entry barrier for relatively small and medium-sized/venture pharmaceutical companies to grow.

The new drug development market is dominated by a small number of global pharmaceutical companies called Big Pharma, which account for 30% or more of the total sales in the entire new drug development field. These global pharmaceutical companies maintain a strategy of applying for patents depending on the development stages of drug candidates to monopolize the economic value of the candidates.

In the pharmaceutical industry, the absolute scale of R&D costs is important for small and medium-sized pharmaceutical companies to ensure competitiveness in new drug development. However, the reality is that the R&D costs of small and medium-sized/venture pharmaceutical companies are much lower than those of global pharmaceutical companies. Accordingly, the gap between global pharmaceutical companies that have ensured exclusive positions in the new drug development market through massive R&D investments and small and medium-sized/venture pharmaceutical companies is gradually widening.

Meanwhile, with the recent development of artificial intelligence, cases of using artificial intelligence in new drug development are increasing. Artificial intelligence is able to effectively reduce the time/cost required for new drug development, quickly select candidates that may have patent issues in the early stages of new drug development, and quickly determine failure before reaching the clinical stage, contributing to increasing the success rate of new drug development. Therefore, cases of applying artificial intelligence to the entire process of new drug development, from selection of candidates to clinical trials, are increasing recently.

New drug development using artificial intelligence is revolutionizing the traditional drug development process, including predicting drug reactivity using public databases based on genomic data, selecting new drug candidates, predicting protein structures, predicting drug toxicity and biological activity, and optimizing clinical trial design.

In addition, although it is very important to identify a target protein with potential as a disease treatment agent in the new drug development stage and to find new drug candidates that bind to the pocket structure of the target protein, the gap between in silico, in vitro, and in vivo experiments is very large. Hence, taking into consideration only the pocket structure of a specific target protein as in the current situation, it is very difficult for a new drug candidate that binds thereto to be approved as a drug after passing cell experiments, animal tests, and clinical trials. In order to drastically increase the success rate of new drug development, known to be 1 in 10,000, it is necessary to overcome the complexity of the stages in which the final new drug product is created and the inconsistency of each stage as much as possible.

Currently, in the process of developing new drugs, AI models are mainly used to select a target protein and then virtually screen novel compounds with high binding affinity to the target protein. However, since humans are known to be composed of approximately 36 trillion cells and have approximately 25,000 genes and 100,000 types of proteins, when a person takes a drug, the drug may bind to countless proteins as well as the target protein, resulting in various side effects and aftereffects, drug efficacy, toxicity, etc.

As such, the possibility of new drug development cannot be drastically improved without considering intracellular biochemical metabolic pathways, intracellular molecular biological mechanisms, etc., such as whether the new drug candidate is able to penetrate the cell membrane, induces structural and functional changes in a specific protein, causes genetic mutations, and has any effect on the activity of signaling materials present in cells, in the process of virtual screening or optimization of novel compounds with high binding affinity to the target protein.

Specifically, in the virtual screening process, it is important to consider the characteristics at multiple levels, not only at the protein level but also at the cellular level, animal level, and human level, and furthermore, in order to confirm the selective specificity of new drug candidates acting as ligands for a target protein, the virtual screening method for all proteins used requires a large amount of computation.

Therefore, it will be necessary to develop technology that generates optimal new drug candidate information using artificial intelligence and big data to effectively reduce the time and cost required for new drug development.

The present invention is based on the above-mentioned necessity, and proposes a method of generating new drug candidate information, which includes identifying hit compounds having selective ligand characteristics only for a target protein, which is a cause of pathogenesis present in cells, from existing compounds using virtual molecular modeling of a protein pocket structure and artificial intelligence, generating information on novel compounds having ligand characteristics similar to the hit compounds for the target protein, and determining, among the novel compounds, novel compounds having predicted binding affinity for the target protein and in vitro properties and in vivo properties that meet preset criteria as new drug candidates. Conventional techniques related thereto are described below.

### CITATION LIST

### Patent Literature

1. Korean Patent No. 10-2296188, “Method and device for designing compound”
2. Korean Patent No. 10-2347108, “New drug candidate prediction device and operation method thereof”
3. Korean Patent No. 10-2558546, “Artificial intelligence learning-based kinase profiling device using multiple sequence information of protein structure and 3D structure descriptor for predicting drug effect and operation method thereof”
4. Korean Patent Application Publication No. 10-2024-0084664, “Method of extracting multi-pharmacophore drug information based on bioactivity data, and analysis device for multi-pharmacophore-based drug screening”

### SUMMARY OF THE INVENTION

An object of the present invention is to provide a method of generating new drug candidate information, which includes identifying hit compounds having selective ligand characteristics only for a target protein, which is a cause of pathogenesis present in cells, from existing compounds using virtual molecular modeling of a protein pocket structure and artificial intelligence, generating information on novel compounds having ligand characteristics similar to the hit compounds for the target protein, and determining, among the novel compounds, novel compounds having predicted binding affinity for the target protein and in vitro and in vivo properties that meet preset criteria as new drug candidates.

In order to accomplish the above object, the present invention provides a method of generating new drug candidate information using virtual molecular modeling of a protein pocket structure, including:
a hit compound search step (S100) of identifying existing compounds having selective ligand characteristics only for a target protein, which is a cause of pathogenesis present in cells, through virtual molecular modeling of a protein pocket structure using artificial intelligence and generating chemical pose information of the identified existing compounds as hit compound information,
an analogue/lead compound search step (S200) of generating information on analogue compounds for novel compounds having ligand characteristics similar to hit compounds for the target protein, identifying, among the analogue compounds, analogue compounds that are predicted to bind to the target protein with binding affinity greater than or equal to a preset value, and generating the identified analogue compound information as lead compound information, and
a new drug search step (S300) of evaluating properties of lead compounds and generating the lead compounds having the properties that meet preset criteria as new drug candidate information.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows a flowchart of the present invention;

FIGS. 2, 3, and 4 show a hit compound search step according to the present invention; and

FIG. 5 shows an analogue/lead compound search step according to the present invention.

### DETAILED DESCRIPTION OF THE INVENTION

Hereinafter, a detailed description will be given of embodiments of the present invention with reference to the attached drawings.

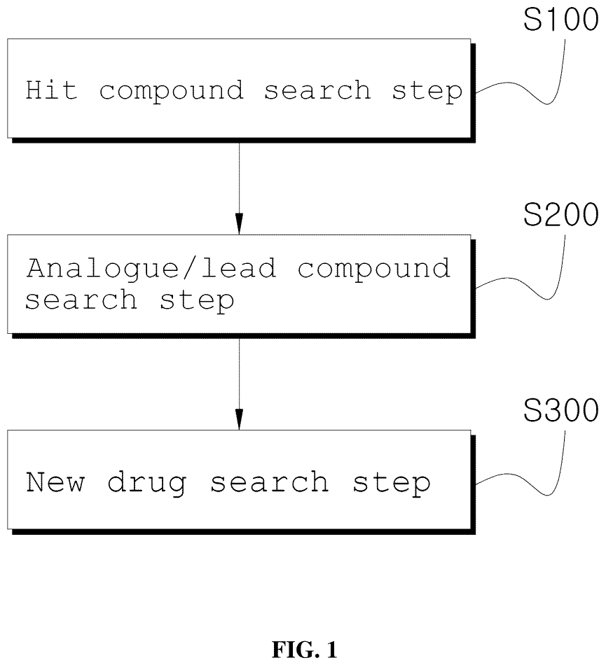

According to the present invention, a method of generating new drug candidate information using virtual molecular modeling of a protein pocket structure includes identifying hit compounds having selective ligand characteristics only for a target protein, which is a cause of pathogenesis present in cells, from existing compounds using virtual molecular modeling of a protein pocket structure and artificial intelligence, generating information on novel compounds having ligand characteristics similar to the hit compounds for the target protein, and determining, among the novel compounds, novel compounds having predicted binding affinity for the target protein and in vitro properties and in vivo properties that meet preset criteria as new drug candidates, thus generating new drug candidate information, thereby enabling effective development of new drugs with relatively low R&D costs and short development time. As shown in FIG. 1, the method of the present invention includes a hit compound search step (S100), an analogue/lead compound search step (S200), and a new drug search step (S300).

Specifically, as shown in FIG. 1, the method of generating new drug candidate information using virtual molecular modeling of a protein pocket structure according to the present invention includes:
a hit compound search step (S100) of identifying existing compounds having selective ligand characteristics only for a target protein, which is a cause of pathogenesis present in cells, through virtual molecular modeling of a protein pocket structure using artificial intelligence and generating chemical pose information of the identified existing compounds as hit compound information,
an analogue/lead compound search step (S200) of generating information on analogue compounds for novel compounds having ligand characteristics similar to the hit compounds for the target protein, identifying, among the analogue compounds, analogue compounds that are predicted to bind to the target protein with binding affinity greater than or equal to a preset value, and generating the identified analogue compound information as lead compound information, and
a new drug search step (S300) of evaluating properties of the lead compounds and generating the lead compounds having the properties that meet preset criteria as new drug candidate information.

The hit compound search step (S100) is a step in which existing compounds having selective ligand characteristics only for a target protein, which is a cause of pathogenesis present in cells, are identified through virtual molecular modeling of a protein pocket structure using artificial intelligence, and the chemical pose information of the identified existing compounds is generated as hit compound information.

In the development of new drugs, it is necessary to identify selective ligand characteristics of known compounds for target proteins (pathogenic proteins) present in cells. During this process, it is necessary to analyze the ligand characteristics for numerous (from hundreds of millions to billions) protein-compound binding pairs between known proteins and known compounds.

However, conventional analysis methods are problematic in that they take too much time to analyze ligand characteristics for numerous protein-compound binding pairs even when using a supercomputer. Therefore, the present invention aims to drastically reduce the time for analyzing ligand characteristics for numerous protein-compound binding pairs through virtual molecular modeling of protein pocket structures using artificial intelligence.

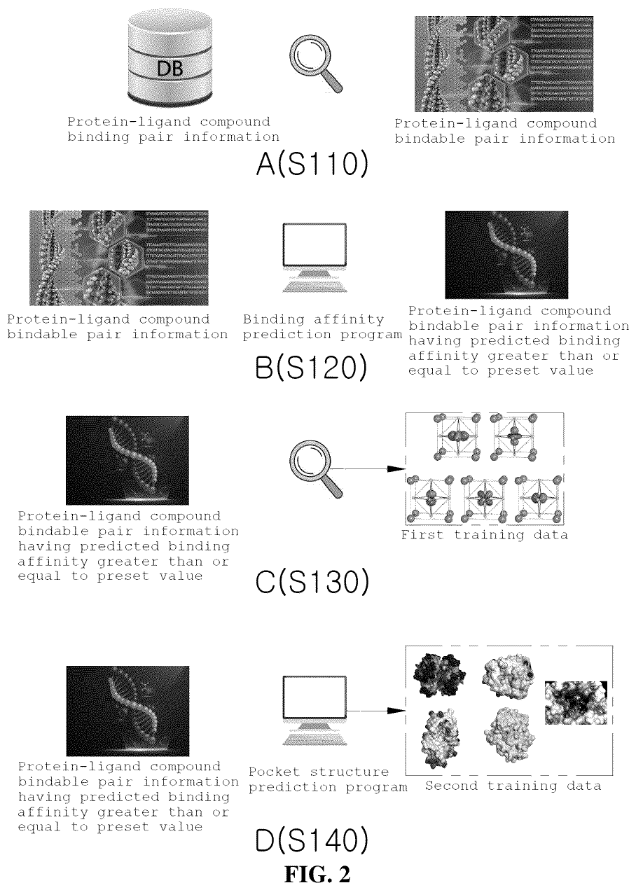

Specifically, as shown in FIGS. 2, 3, and 4, the hit compound search step (S100) includes:
a first step (S110) of generating information on protein-ligand compound bindable pairs, which are bindable pairs between known proteins and known ligand compounds, using known protein-ligand compound binding pair information,
a second step (S120) of predicting binding affinity between a protein and a ligand compound for each of the protein-ligand compound bindable pairs and generating information on protein-ligand compound bindable pairs having the predicted binding affinity greater than or equal to a preset value,
a third step (S130) of generating first training data on binding relationship characteristics between proteins and ligand compounds constituting the protein-ligand compound bindable pairs having the predicted binding affinity greater than or equal to a preset value,
a fourth step (S140) of generating second training data on the pocket structure of each of the proteins constituting the protein-ligand compound bindable pairs having the predicted binding affinity greater than or equal to a preset value,
a fifth step (S150) of generating a binding affinity prediction model for each known protein, capable of predicting binding affinity between a protein and a compound when the compound binds to the protein, through virtual molecular modeling of the protein pocket structure by artificial intelligence 10 trained using the first and second training data, and
a sixth step (S160) of identifying existing compounds that act as ligands for the target protein but do not act as ligands for proteins other than the target protein using the generated binding affinity prediction model for each known protein and the chemical pose information of existing compounds, and generating the chemical pose information of the identified existing compounds as hit compound information.

As shown in A of FIG. 2, the first step (S110) is a step of generating information on protein-ligand compound bindable pairs, which are bindable pairs between known proteins and known ligand compounds, using known protein-ligand compound binding pair information.

There are about 20,000 pieces of known protein-ligand compound binding pair information, and from the known protein-ligand compound binding pair information, information on about 20,000 proteins and about 20,000 ligand compounds may be obtained, and also, information on protein-ligand compound bindable pairs (20,000×20,000=about 400 million binding pairs), which are bindable pairs between about 20,000 proteins and about 20,000 ligand compounds, is obtained.

For example, information on protein Nos. 1 to 19,443 and information on ligand compound Nos. 1 to 19,443 are obtained from known protein-ligand compound binding pair information and then cross-linked, generating information on 378,030,249 protein-ligand compound bindable pairs.

As shown in B of FIG. 2, the second step (S120) is a step of predicting binding affinity between a protein and a ligand compound for each of the protein-ligand compound bindable pairs and generating information on protein-ligand compound bindable pairs having the predicted binding affinity greater than or equal to a preset value.

For example, when information on 378,030,249 protein-ligand compound bindable pairs is generated in the first step (S110), binding affinity between the protein and the ligand compound is predicted for each of the generated 378,030,249 protein-ligand compound bindable pairs using a conventional binding affinity prediction program (rDock, AutoDock, FlexAID, etc.), after which protein-ligand compound bindable pairs having the predicted binding affinity greater than or equal to a preset value (in which the absolute value of predicted binding affinity score is 4 or higher) are classified and identified, generating information on protein-ligand compound bindable pairs having the predicted binding affinity greater than or equal to a preset value.

As shown in C of FIG. 2, the third step (S130) is a step of generating first training data on the binding relationship characteristics between proteins and ligand compounds constituting the protein-ligand compound bindable pairs having the predicted binding affinity greater than or equal to a preset value.

The first training data are generated by identifying the binding relationship characteristics between the proteins and the ligand compounds for the protein-ligand compound bindable pairs having the predicted binding affinity greater than or equal to a preset value generated in the second step (S120). The binding relationship characteristics between the proteins and the ligand compounds include the type, number, binding structure, and binding affinity of protein atoms in a binding relationship with the ligand compound, and thus the first training data include information on the type, number, binding structure, and binding affinity of protein atoms in a binding relationship with the ligand compound.

For example, if the number of bindable pairs having the predicted binding affinity greater than or equal to a preset value is 50,000,000, the type, number, binding structure, and binding affinity of protein atoms around the protein pocket, which are the binding relationship characteristics between the protein and the ligand compound for each of the 50,000,000 protein-ligand compound bindable pairs, are identified using a supercomputer. When the binding relationship characteristics between the protein and the ligand compound, such as the type, number, binding structure, and binding affinity of the protein atoms around the protein pocket, are identified, the first training data on the binding relationship characteristics between the proteins and the ligand compounds constituting the protein-ligand compound bindable pairs having the predicted binding affinity greater than or equal to a preset value, are generated.

As shown in D of FIG. 2, the fourth step (S140) is a step of generating second training data on the pocket structure of each of the proteins constituting the protein-ligand compound bindable pairs having the predicted binding affinity greater than or equal to a preset value.

For the protein-ligand compound bindable pairs having the predicted binding affinity greater than or equal to a preset value generated in the second step (S120), the pocket structures of the proteins constituting the protein-ligand compound bindable pairs having the predicted binding affinity greater than or equal to a preset value (e.g., the absolute value of predicted binding affinity score of 4 or higher) may be identified using a conventional pocket structure prediction program (e.g., CLAPE, DeepProSite, etc.). Here, the protein pocket is a protein region to which a ligand compound is bound, and the identified protein pocket structure may be related to the structural shape or form of the pocket.

For example, if the number of bindable pairs having the predicted binding affinity greater than or equal to a preset value is 50,000,000, the pocket structures of proteins constituting the 50,000,000 protein-ligand compound bindable pairs are identified using a pocket structure prediction program (e.g., CLAPE, DeepProSite, etc.). When the pocket structures of the proteins are identified, the second training data on the pocket structures of the proteins constituting the bindable pairs having the predicted binding affinity greater than or equal to a preset value are generated.

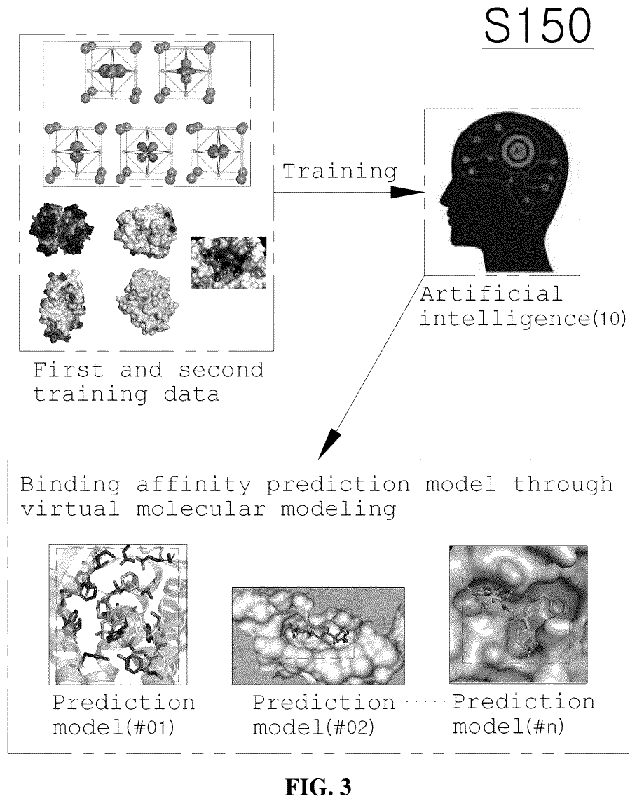

As shown in FIG. 3, the fifth step (S150) is a step of generating a binding affinity prediction model for each known protein, capable of predicting binding affinity between a protein and a compound when the compound binds to the protein, through virtual molecular modeling of the protein pocket structure by the artificial intelligence 10 that was trained using the first and second training data.

Specifically, by the artificial intelligence 10 in the fifth step (S150), pocket structure characteristics of the protein pocket, which is a region to which the ligand compound is bound, are learned for each protein using the second training data, and mutual binding relationship characteristics between the protein atoms around the pocket and the ligand compound atoms in a binding relationship with the corresponding protein atoms are learned using information on the type, number, binding structure, and binding affinity of the protein atoms in a binding relationship with the ligand compound included in the first training data.

Also, using the learning results, the characteristics of virtual atoms that replace real protein atoms around the pocket are defined, and virtual molecular modeling of the protein pocket as a virtual molecule composed of virtual atoms having the defined characteristics is performed for each known protein, so the protein pocket is modeled as a virtual molecule, and a binding affinity prediction model capable of predicting binding affinity between a protein and a compound when the compound binds to the protein is created for each known protein.

The characteristics of the virtual atoms are binding to ligand compound atoms like real protein atoms when real ligand compound atoms bind to real protein atoms around the pocket.

By the artificial intelligence, the pocket structure characteristics of the protein pocket, which is a region to which the ligand compound is bound, are learned using the second training data. The second training data are related to the pocket structure of the protein bound to the ligand compound, for example, the structural shape or form of the pocket. Using the second training data, the artificial intelligence learns what structural characteristics of the pocket structure of the protein bound to the ligand compound are responsible for binding to the ligand compound.

In addition, by the artificial intelligence, mutual binding relationship characteristics between the protein atoms around the pocket and the ligand compound atoms that are in a binding relationship with the corresponding protein are learned using the first training data. The first training data include information on the type, number, binding structure, and binding affinity of the protein atoms that are in a binding relationship with the ligand compound, and the artificial intelligence learns what binding characteristics the protein atoms and the ligand compound atoms that are in a binding relationship with the corresponding protein are bound to using the first training data.

Then, using the learning results, the characteristics of virtual atoms that replace real protein atoms around the pocket are defined by the artificial intelligence. The characteristics of the virtual atoms are binding to ligand compound atoms like real protein atoms when real ligand compound atoms bind to real protein atoms around the pocket.

When the characteristics of the virtual atoms are defined, by the artificial intelligence, a virtual molecule composed of virtual atoms having the defined characteristics is constructed and virtual molecular modeling of the protein pocket as the constructed virtual molecule is performed. Virtual molecular modeling of the protein pocket means replacing a protein pocket with a virtual molecule composed of virtual atoms having defined characteristics, and virtual molecular modeling of the protein pocket is performed for each known protein.

Using the virtual molecular modeling of the protein pocket performed for each known protein, the protein pocket is modeled as a virtual molecule, and a binding affinity prediction model capable of predicting binding affinity between a protein and a compound when the compound binds to the protein is generated for each known protein.

In the binding affinity prediction model generated for each known protein, when chemical pose information of a specific compound is given as an input value, a binding affinity prediction value between the protein and the specific compound is output as an output value.

When inputting the chemical pose information of existing compounds as the input value into the binding affinity prediction model for each known protein in the fifth step (S150), binding affinity between known proteins and known compounds may be rapidly predicted, thereby solving problems with conventional new drug development processes in which too much time is required to analyze ligand characteristics for numerous protein-compound binding pairs.

The sixth step (S160) is a step of identifying existing compounds that act as ligands for the target protein but do not act as ligands for proteins other than the target protein using the generated binding affinity prediction model for each known protein and the chemical pose information of existing compounds, and generating the chemical pose information of the identified existing compounds as hit compound information.

A new drug must have selective ligand characteristics that act as a ligand for the target protein and do not act as a ligand for proteins other than the target protein. Accordingly, new drug candidates must also have selective ligand characteristics that do not act as ligands for proteins other than the target protein.

In the analogue/lead compound search step (S200) described below according to the present invention, analogue compounds are first identified and then, among the identified analogue compounds, lead compounds to be used as new drug candidates are identified. The analogue compounds are derived from the hit compound information generated in step (S160), and thus the hit compounds must also have selective ligand characteristics that act as ligands for the target protein and do not act as ligands for proteins other than the target protein. The sixth step (S160) is performed to identify hit compounds having selective ligand characteristics.

Specifically, the sixth step (S160) is characterized in that the chemical pose information of existing compounds extracted from compound DB is individually input into the generated binding affinity prediction model for each known protein, generating binding affinity prediction information between existing compounds and known proteins, and using the generated binding affinity prediction information between existing compounds and known proteins, a reference binding affinity ((−) binding affinity having the greatest absolute value among binding affinities between the target protein and existing compounds) is determined, and then existing compounds corresponding to hit identification conditions are selected as hit compounds, and chemical pose information of the existing compounds selected as the hit compounds is generated as hit compound information. The hit identification conditions are such that the absolute value of binding affinity to other proteins is less than a first set % of the absolute value of reference binding affinity, and the sum of the absolute values of binding affinities to other proteins within a second set % range of the reference binding affinity is less than or equal to three times the absolute value of reference binding affinity.

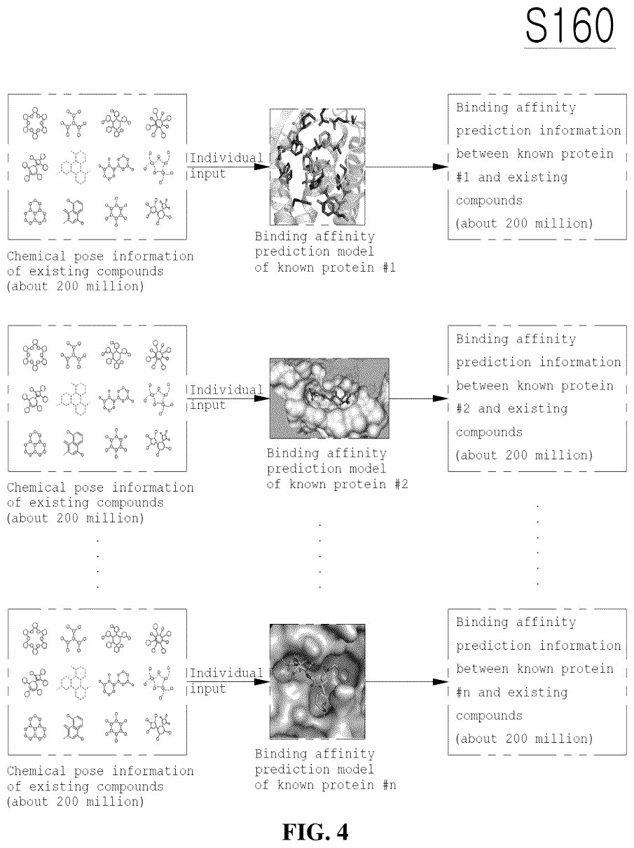

Referring to FIG. 4, for example, if there are 19,443 types of known proteins and 220,799,302 types of existing compounds that may be extracted from compound DB, the chemical pose information of 220,799,302 existing compounds is individually input into the generated binding affinity prediction model for each of 19,443 known proteins, generating binding affinity prediction information between existing compounds and known proteins.

For example, as shown in FIG. 4, when the chemical pose information of existing compounds (about 220,799,302 compounds) extracted from compound DB is individually input into the binding affinity prediction model of known protein #1, the binding affinity prediction model of protein #1 generates binding affinity prediction information between known protein #1 and existing compounds (about 220,799,302 compounds). The binding affinity prediction information between known protein #1 and existing compounds (about 220,799,302 compounds) includes about 220,799,302 binding affinity prediction values ((a binding affinity prediction value of protein #1 and existing compound #1), (a binding affinity prediction value of protein #1 and existing compound #2), (a binding affinity prediction value of protein #1 and existing compound #3), . . . , and (a binding affinity prediction value of protein #1 and existing compound #220,799,302)).

In addition, when the chemical pose information of existing compounds (about 220,799,302 compounds) extracted from compound DB is individually input into the binding affinity prediction model of known protein #2, the binding affinity prediction model of protein #2 generates binding affinity prediction information between known protein #2 and existing compounds (about 220,799,302 compounds). The binding affinity prediction information between known protein #2 and existing compounds (about 220,799,302 compounds) includes about 220,799,302 binding affinity prediction values ((a binding affinity prediction value of protein #2 and existing compound #1), (a binding affinity prediction value of protein #2 and existing compound #2), (a binding affinity prediction value of protein #2 and existing compound #3), . . . , and (a binding affinity prediction value of protein #2 and existing compound #220,799,302)).

In the same way as above, by individually inputting the chemical pose information of existing compounds (about 220,799,302 compounds) extracted from compound DB into the binding affinity prediction model of last known protein #19,443, binding affinity prediction information between existing compounds and known proteins is generated.

When the binding affinity prediction information between existing compounds and known proteins is generated, the reference binding affinity is determined using the generated binding affinity prediction information between existing compounds and known proteins.

The binding affinity between existing compounds and known proteins may be (+) binding affinity or (−) binding affinity. (+) binding affinity means that binding affinity between a compound and a protein is weak, and the larger the absolute value, the less the binding affinity. In contrast, (−) binding affinity means that binding affinity between a compound and a protein is strong, and the larger the absolute value, the more the binding affinity.

The reference binding affinity indicates the (−) binding affinity with the greatest absolute value among the binding affinities of the target protein and existing compounds. For example, using the binding affinity prediction information between existing compounds and known proteins, if the absolute value 18 of binding affinity is the greatest among the (−) binding affinities of the target protein and existing compounds, −18 is determined as the reference binding affinity.

After determining the reference binding affinity, existing compounds corresponding to the hit identification conditions are selected as hit compounds. As such, the hit identification conditions are such that each of the absolute values of binding affinities to proteins other than the target protein is less than a first set % of the absolute value of reference binding affinity, and the binding affinities to proteins other than the target protein are within a second set % range of the reference binding affinity, in which the sum of the absolute values of binding affinities to other proteins is less than or equal to three times the absolute value of reference binding affinity. Here, the upper limit of the second set % range is less than the first set %, and the first set % and the second set % range may be arbitrarily adjusted.

When the reference binding affinity is determined, whether all existing compounds meet the hit identification conditions is determined.

For example, hit identification is described assuming that the determined reference binding affinity is −18, the first set % is 90%, and the second set % range is 30-20%.

First, existing compounds in which the absolute value of binding affinity to proteins other than the target protein is greater than or equal to 16.2, which is 90% of the absolute value 18 of reference binding affinity −18 (existing compounds having (−) binding affinity in which the absolute value of binding affinity to other proteins is greater than 16.2) are excluded from hit compound candidates. Briefly, existing compounds having (−) binding affinity in which the binding affinity to at least one other protein is 90% or more of the absolute value of reference binding affinity are excluded from hit compound candidates.

In addition, existing compounds, in which each of the absolute values of binding affinities to proteins other than the target protein is less than 16.2, which is 90% of the absolute value 18 of reference binding affinity (having (−) binding affinity but the absolute value of binding affinity to other proteins of less than 16.2), the binding affinities to proteins other than the target protein are in the range of −5.4to −3.6 , which is 30-20% of the reference binding affinity −18, and the sum of the absolute values of binding affinities to other proteins is less than or equal to 54, which is three times the absolute value 18 of reference binding affinity, are selected as hit compounds.

For example, when there are existing compounds A and B, each of which has an absolute value of binding affinity to proteins other than the target protein of less than 16.2, which is 90% of the absolute value of reference binding affinity, and in which binding affinities to proteins other than the target protein are in the range of −5.4to −3.6 , which is 30-20% of the reference binding affinity, if the sum of the absolute values of binding affinities of existing compound A to other proteins is 40 (less than or equal to 54, which is three times the absolute value 18 of reference binding affinity), existing compound A is selected as a hit compound, and if the sum of the absolute values of binding affinities of existing compound B to other proteins is 60 (greater than or equal to 54, which is three times the absolute value 18 of reference binding affinity), existing compound B is not selected as a hit compound.

Through the above process, hit compounds are selected from existing compounds, and the chemical pose information of existing compounds selected as the hit compounds is generated as hit compound information (less than or equal to 54, which is three times the absolute value 18 of reference binding affinity).

The analogue/lead compound search step (S200) is a step of generating information on analogue compounds for novel compounds having ligand characteristics similar to the hit compounds for the target protein, identifying, among the analogue compounds, analogue compounds that are predicted to bind to the target protein with binding affinity greater than or equal to a preset value, and generating the identified analogue compound information as lead compound information.

Specifically, the analogue/lead compound search step (S200) is characterized by generating, for each of the hit compounds derived in the hit compound search step (S100), chemical pose information of novel compounds that do not exist before and have ligand binding characteristics similar to the hit compounds derived in the hit compound search step (S100) for the target protein, generating the chemical pose information of novel compounds thus obtained as analogue compound information, predicting the binding affinity between the novel compounds generated as the analogue compounds and the target protein using a binding affinity prediction program, and generating the chemical pose information of the novel compounds having the predicted binding affinity greater than or equal to a preset value as lead compound information. Here, the ligand binding characteristics include hydrogen bonding, ionic bonding, and pi-pi interaction.

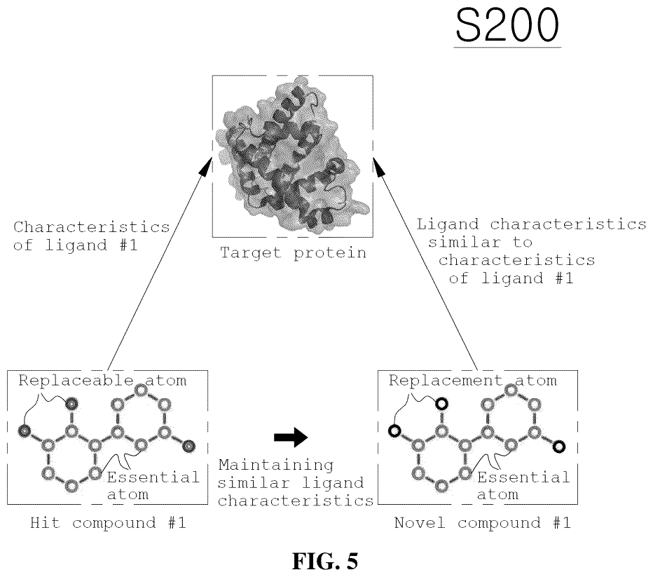

For example, among the hit compounds derived in the hit compound search step (S100), for hit compound #1, chemical pose information of novel compound #1 that does not exist before and has ligand binding characteristics (including hydrogen bonding, ionic bonding, and pi-pi interaction) similar to hit compound #1 for the target protein is generated. For example, as shown in FIG. 5, among the atoms of hit compound #1, atoms (replaceable atoms) that maintain the ligand binding characteristics (including hydrogen bonding, ionic bonding, and pi-pi interaction) of the hit compound for the target protein even when replaced with other atoms are determined, and by replacing the determined atoms with other replacement atoms, chemical pose information of novel compound #1 that does not exist before and has ligand binding characteristics similar to hit compound #1 for the target protein is generated.

In the same way, for hit compound #2, chemical pose information of novel compound #2 that does not exist before and has ligand binding characteristics (including hydrogen bonding, ionic bonding, and pi-pi interaction) similar to hit compound #2 for the target protein is generated, and by performing this process up to the last hit compound, the chemical pose information of n novel compounds corresponding to all hit compounds (n hit compounds) derived in the hit compound search step (S100) is generated as analogue compound information.

When the analogue compound information is generated, the binding affinity between all novel compounds corresponding to the analogue compound information and the target protein is predicted using a binding affinity prediction program (rDock, AutoDock, FlexAID, etc.), and the chemical pose information of the novel compounds having the predicted binding affinity greater than or equal to a preset value (e.g., an absolute value of predicted binding affinity score of 6 or higher) is generated as lead compound information.

The new drug search step (S300) is a step of evaluating properties of the lead compounds and generating the lead compounds having the properties that meet preset criteria as new drug candidate information. The properties of the lead compounds include in vitro properties and in vivo properties.

The in vitro properties include fat/water solubility, cell membrane permeability (Caco-2 and MDCK cell permeability), degree of CYP450 enzyme activity inhibition, metabolic stability, plasma stability, CYP expression induction by the drug, cytotoxicity, cell activity, efficacy evaluated by the IC50 value, which is a quantitative measurement value indicating the amount of a lead compound required to inhibit a given biological process or biological component by 50% in vitro, tolerability, side effects, and drug stability in the body, and the in vivo properties include the half lethal dose of test animals, change in the concentration of the lead compound over time, time to reach peak blood concentration (Tmax), peak blood concentration (Tmax) and half-life (T1/2) of the lead compound, whether the lead compound passes through BBB, and BBB permeability.

The lead compounds having the in vitro and in vivo properties that meet preset criteria are generated as new drug candidate information.

As is apparent from the foregoing, the present invention is capable of generating new drug candidate information by identifying hit compounds having selective ligand characteristics only for a target protein, which is a cause of pathogenesis present in cells, from existing compounds using virtual molecular modeling of a protein pocket structure and artificial intelligence, generating information on novel compounds having ligand characteristics similar to the hit compounds for the target protein, and determining, among the novel compounds, novel compounds having predicted binding affinity for the target protein and in vitro properties and in vivo properties that meet preset criteria as new drug candidates, thereby enabling effective development of new drugs with relatively low R&D costs and short development time.

Although the technical spirit of the present invention has been described above with reference to the attached drawings, this is merely illustrative of preferred embodiments of the present invention and is not to be construed as limiting the present invention. It will be obvious that the scope of the present invention is not limited to the embodiments and also includes modifications made within the technical spirit and scope of the present invention by a person of ordinary skill in the art.

What is claimed is:

1. A method of generating new drug candidate information using virtual molecular modeling of a protein pocket structure, comprising:
a hit compound search step (S100) of identifying existing compounds having selective ligand characteristics for a target protein, which is a cause of pathogenesis present in cells, through virtual molecular modeling of a protein pocket structure using artificial intelligence and generating chemical pose information of the identified existing compounds as hit compound information;
an analogue/lead compound search step (S200) of generating information on analogue compounds for novel compounds having ligand characteristics similar to hit compounds for the target protein, identifying, among the analogue compounds, analogue compounds that are predicted to bind to the target protein with binding affinity greater than or equal to a preset value, and generating the identified analogue compound information as lead compound information; and
a new drug search step (S300) of evaluating properties of lead compounds and generating the lead compounds having the properties that meet preset criteria as new drug candidate information.

2. The method of claim 1, wherein the hit compound search step (S100) comprises:
a first step (S110) of generating information on protein-ligand compound bindable pairs, which are bindable pairs between known proteins and known ligand compounds, using known protein-ligand compound binding pair information,
a second step (S120) of predicting binding affinity between a protein and a ligand compound for each of the protein-ligand compound bindable pairs and generating information on protein-ligand compound bindable pairs having the predicted binding affinity greater than or equal to a preset value,
a third step (S130) of generating first training data on binding relationship characteristics between proteins and ligand compounds constituting the protein-ligand compound bindable pairs having the predicted binding affinity greater than or equal to a preset value,
a fourth step (S140) of generating second training data on a pocket structure of each of the proteins constituting the protein-ligand compound bindable pairs having the predicted binding affinity greater than or equal to a preset value,
a fifth step (S150) of generating a binding affinity prediction model for each known protein, capable of predicting binding affinity between a protein and a compound when the compound binds to the protein, through virtual molecular modeling of the protein pocket structure by artificial intelligence (10) trained using the first and second training data, and
a sixth step (S160) of identifying existing compounds that act as ligands for the target protein but do not act as ligands for proteins other than the target protein using the generated binding affinity prediction model for each known protein and chemical pose information of existing compounds, and generating chemical pose information of the identified existing compounds as hit compound information.

3. The method of claim 2, wherein:
the first training data comprise information on type, number, binding structure, and binding affinity of protein atoms in a binding relationship with the ligand compound, and
by the artificial intelligence (10) in the fifth step (S150),
using the second training data, pocket structure characteristics of a protein pocket, which is a region to which the ligand compound is bound, are learned for each protein, and using the information on the type, number, binding structure, and binding affinity of protein atoms in a binding relationship with the ligand compound included in the first training data, mutual binding relationship characteristics between protein atoms around the pocket and ligand compound atoms in a binding relationship with the protein atoms are learned, and
using the learning results, characteristics of virtual atoms that replace real protein atoms around the pocket are defined, and virtual molecular modeling of the protein pocket as a virtual molecule composed of the virtual atoms having the defined characteristics is performed for each known protein, so the protein pocket is modeled as the virtual molecule, and a binding affinity prediction model capable of predicting binding affinity between a protein and a compound when the compound binds to the protein is generated for each known protein,
in which the characteristics of the virtual atoms are binding to ligand compound atoms like real protein atoms when real ligand compound atoms bind to real protein atoms around the pocket.

4. The method of claim 2, wherein:
in the sixth step (S160),
chemical pose information of existing compounds extracted from compound DB is individually input into the generated binding affinity prediction model for each known protein, generating binding affinity prediction information between existing compounds and known proteins, and using the generated binding affinity prediction information between existing compounds and known proteins, a reference binding affinity ((−) binding affinity with a greatest absolute value among binding affinities of the target protein and existing compounds) is determined, after which existing compounds corresponding to hit identification conditions are selected as hit compounds, and chemical pose information of the existing compounds selected as the hit compounds is generated as hit compound information,
in which the hit identification conditions are such that each of absolute values of binding affinities to proteins other than the target protein is less than a first set % of an absolute value of reference binding affinity, and binding affinities to proteins other than the target protein are within a second set % range of the reference binding affinity, in which a sum of the absolute values of binding affinities to other proteins is less than or equal to three times the absolute value of reference binding affinity, and
an upper limit of the second set % range is less than the first set %.

5. The method of claim 1, wherein:
in the analogue/lead compound search step (S200),
chemical pose information of novel compounds that do not exist before and have ligand binding characteristics similar to the hit compounds derived in the hit compound search step (S100) for the target protein is generated for each of the hit compounds derived in the hit compound search step (S100), and the chemical pose information of the novel compounds thus obtained is generated as analogue compound information, and
binding affinity between the novel compounds generated as analogue compounds and the target protein is predicted using a binding affinity prediction program, and chemical pose information of the novel compounds having the predicted binding affinity greater than or equal to a preset value is generated as lead compound information,
in which the ligand binding characteristics comprise hydrogen bonding, ionic bonding, and pi-pi interaction.

6. The method of claim 1, wherein the properties of the lead compounds in the new drug search step (S300) comprise in vitro properties and in vivo properties.
